(19) United States
(12) Patent Application Publication
(10) Pub. No.: US 2019/0083390 A1
(43) Pub. Date: Mar. 21, 2019

(54) RicinoLAX Rectal Stimulant Laxative

(71) Applicant: ALAN DALE RHOADS, ORELAND, PA (US)

(72) Inventor: Alan Dale Rhoads, ORELAND, PA (US)

(21) Appl. No.: 15/709,537

(22) Filed: Sep. 20, 2017

(51) Int. Cl. A61K 9/00; A61K 31/201; A61K 36/47

(52) U.S. Cl. CPC A61K 9/0031; A61K 36/47; A61K 31/201

(57) ABSTRACT

Castor Oil, when administered as a rectal laxative, acts solely as a lubricant.

RicinoLAX provides the unique properties of Castor Oil that has been converted (it is presently believed) to Ricinoleic Acid by natural enzymes.

This patent, and its required artificial enzymes, and chemicals, have yet to become technically understood and resolved. This patent Author is unaware of any methods that are yet devised to produce RicinoLAX.

RicinoLAX provides an alternate remedy and a substitute for oral Castor Oil.

RicinoLAX avoids the distasteful and nauseating taste of oral Castor Oil.

The study of stomach and small intestines enzymes are ongoing with little definitive identification of how Castor Oil is actually converted, nor how it plays in stimulating laxative results. It is unknown whether Castor Oil's Ricinoleic Acid is the stimulant directly working on the large intestines, or whether it is absorbed into the blood stream to afford its laxative properties.

### CROSS REFERENCE TO RELATED APPLICATIONS

n/a

### STATEMENT OF FEDERALLY SPONSORED RESEARCH

n/a

### THE NAMES OF THE PARTIES TO A JOINT RESEARCH OR DEVELOPMENT

n/a

### REFERENCE TO A “SEQUENCE LISTING”

n/a

### BACKGROUND OF THE INVENTION

This patent for RicinoLAX Rectal Stimulant Laxative falls under the classification of Pharmaceutical process of the conversion of an existing product, Castor Oil. RicinoLAX Rectal Stimulant Laxative is further classified as a non-provisional Utility patent and the patent Author's status is designated as being a Micro-entity. The patent avoids the requirement of taking an oral dose of Castor Oil. Scientists are still guessing how Castor Oil actually provides peristalsis activity in the bowel.

### BRIEF SUMMARY OF THE INVENTION

The objective of this patent is to provide an alternative stimulant laxative application to the oral dosing using Castor Oil and yet provide identical results. This patent would be classified as CCL/514/892.

### BRIEF DESCRIPTION OF THE SEVERAL VIEWS OF THE DRAWING (IF ANY)

n/a

### DETAILED DESCRIPTION OF THE INVENTION

Because a rectal dose of Castor Oil cannot induce a stimulant laxative effect, but exhibits a lubricant effect it cannot provide peristalsis. RicinoLAX Rectal Stimulant Laxative has the desired results. However, RicinoLAX has yet to be created. Even its process using artificial enzymes are beyond today's technology and science. Therefore, it is impossible to describe its Detailed Description.

### SEQUENCE LISTING (IF ANY)

n/a

I claim:

1. RicinoLAX Rectal Stimulant Laxative, shall be derived from pure, FDA-approved Castor Oil, (pharmaceutical grade), the source material for this patent.

2. All artificial enzymes, and all unique and previously unknown chemicals, required for the creation of RicinoLAX, are protected by this patent.

3. RicinoLAX provides purgative peristalsis action resulting in a serious remedy for constipation, as a rectal laxative, or as a complete rectal evacuant.

4. RicinoLAX as developed and produced by the results of the artificial enzymes and unique chemicals, has the same efficacy as an oral dose of Castor Oil that:
has orally entered the gastrointestinal tract of the human body,
and has been converted by the bodily processes to become a strong stimulant laxative in the large intestine.

5. The rectal stimulant laxative, RicinoLAX, is a liquid enema product having a standardized shelf life with expiration date exceeding three years, pending upon storage temperature and conditions.

6. The standardized dose of RicinoLAX enema is contained in a disposable plastic enema container, complete with a silicon rubber rectal tube of sufficient length to reach the distribution area within the large intestine.

7. No additives, modifications, or other modifying mechanisms, except essential preserving additives, shall be incorporated.

8. All Royalties from this patent are owned by the patent Author and his heirs upon his decease.

9. The artificial enzyme(s) and any unique chemical(s) that are required to convert Castor Oil into the RicinoLAX Rectal Stimulant Laxative, are prohibited from being used for any other purposes.
